(19) United States
(12) Patent Application Publication
(10) Pub. No.: US 2012/0318606 A1
(43) Pub. Date: Dec. 20, 2012

(54) Device for stethoscopes

(76) Inventor: Shuo S. Wang, Sierra Madre, CA (US)

(21) Appl. No.: 13/161,560

(22) Filed: Jun. 16, 2011

(51) Int. Cl. A61B 7/02

(52) U.S. Cl. 181/131

(57) ABSTRACT

A single use and disposable device which can consist of thin material that can be applied to and be removed from the auscultation portion of stethoscopes in order to prevent transmissions of potential pathogens.

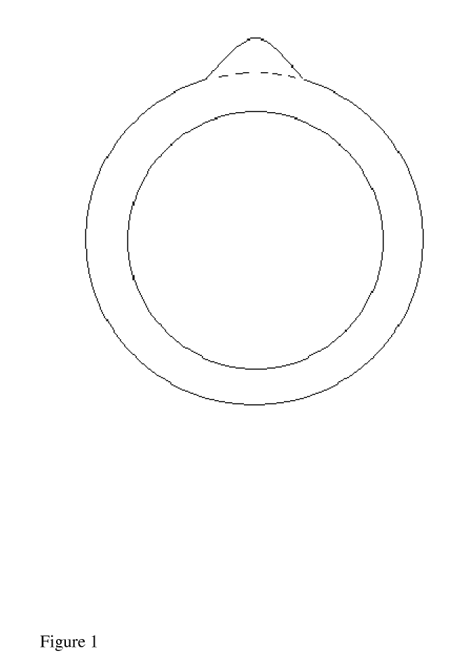

### BACKGROUND OF INVENTION

Medical stethoscopes are documented carriers of pathogens which can be transmitted from one patient to another patient as they are used from room to room without being cleaned. This applies to not only physicians but also nurses, respiratory technicians, and other health care professionals.

### SUMMARY

We describe a single use, disposable device consisting of thin material such as thin plastic film that can cover the auscultation aspect of stethoscopes and through its use can prevent transmissions of dangerous pathogens. In this current environment of increasing healthcare costs reducing preventable illnesses can serve to reduce healthcare costs. Safe, strong, and secure barriers which can prevent the transmission of infections from patient to patient is described and intended in the present invention. The need for prevention of transmission of virulent strains of pathogens is especially needed for intensive care unit patients, burn patients. Furthermore, current methods for prevention of transmission include usage of disposable stethoscopes which have poor sound transmission properties.

Materials such as thin plastic film as they are thin, safe, and secure material can be used in the present manifestation.

A potential manifestation would involve a collection of individual single use devices in a suitably shaped dispenser which users can peel a single use plastic thin film and attach to their stethoscopes for use. This arrangement allows for rapid dispensing of clean and single sheet of material. Other manifestation of use for covers with sleeves would involve dispensers which allow users to dispense and insert their stethoscope into and thus cover their stethoscopes.

### DESCRIPTION OF DRAWING

Potential manifestation of device for stethoscopes with possible extruded edge.

1. device which is used as a cover for stethoscopes that is single use and disposable
device which consists of material that is thin in thickness in the area that is directly in contact with the auscultation aspect of stethoscopes
device which may have outer area that consists of material which maybe of different quality than inner material that is in contact with the auscultation aspect of stethoscopes
device which can be of different shapes but of sufficient surface area of thin material to cover auscultation aspect of stethoscopes
device whose area that is in contact with auscultation aspect of stethoscopes consists of material that has good sound transmission properties
device whose area that is in contact with auscultation aspect of stethoscopes consists of material that has good adherence properties
device whose area is in contact with the auscultation aspect of stethoscope approximates the shape and size of the auscultation aspect of the stethoscope
device whose manifestation may be a single sheet to cover the auscultation aspect of the stethoscope only or may have a sleeve like cover attached to the single sheet area that is in contact with the auscultation aspect of the stethoscope to enclose the auscultation area of the stethoscope and lengths of the rubber tube which leads to the auscultation of the stethoscope
device whose manifestation maybe be single sheet to cover the auscultation aspect of the stethoscope only or may have additional area that is attached to the single sheet area that is in contact with the auscultation aspect of the stethoscope and enclose the entire auscultation aspect of the stethoscope.
device whose material has thin and safe qualities
device which can be taken off from surface which it adheres or clings to
device can be arranged in a easily dispensable manner so that is can be conveniently dispensed for use
device can be arranged such that material can be dispensed without touching the surface area in a handsfree manner
device whose material can be of such quality as to be antibacterial
device can be easily arranged such to be portable and be dispensed by individuals
